(19) United States
(12) Patent Application Publication
(10) Pub. No.: US 2006/0100187 A1
(43) Pub. Date: May 11, 2006

(54) Synergistic combinations of macrolide t-cell modulator or immunosuppressant and a retinoid

(76) Inventors: Maximilian Grassberger, Wien (AT); Stefan Hirsch, Lorrach (DE); Friedrich Karl Mayer, Oberwil (CH); Josef Gottfried Meingassner, Perchtoldsdorf (AT); Carle Paul, Mulhouse (FR); Nabila Sekkat, Basel (CH); Anton Stutz, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/550,358

(22) PCT Filed: Apr. 2, 2004
(86) PCT No.: PCT/EP04/03511

§ 371 Date: Sep. 23, 2005

(30) Foreign Application Priority Data
Apr. 4, 2003 (GB) 0307864.9

(51) Int. Cl. A61K 31/203; A61K 31/33

(52) U.S. Cl. 514/183; 514/559

(57) ABSTRACT

Additive or synergistic combinations of a macrolide T-cell immunomodulator or immunosuppressant such as 33-epichloro-33-desoxyascomycin and a retinoid, such as etretinate, isotretinoin or tazarotene, optionally with a further pharmaceutically active agent, especially an antibacterial, are provided, which are useful in particular in the treatment of dermatological diseases such as eczema, atopic dermatitis, acne, psoriasis, skin aging, sun damage, post-peel erythema and stretch marks.

The invention relates to pharmaceutical compositions, for use in particular in the treatment of skin diseases. It concerns a pharmaceutical composition comprising a macrolide T-cell immunomodulator or immunosuppressant and a retinoid.

It has now been found that, surprisingly, macrolide T-cell immunomodulators and immunosuppressants, when used in combination with retinoids, act additively or synergistically, resulting in a potentiation of pharmacological activity, such that effective beneficial, especially antipsoriatic and anti-acne activity and ability to treat e.g. skin aging, sun damage, post-peel erythema and stretch marks is seen upon co-administration at dosages which would be well below the effective dosages administered individually; the tolerability of, in particular, retinoids is improved, by reducing the side effects associated with retinoid usage (skin irritation, erythema), thereby increasing overall patient acceptance, tolerability and ultimate efficacy.

The invention thus concerns novel pharmaceutical compositions comprising a macrolide T-cell immunomodulator or immunosuppressant in association or combination with a retinoid, hereinafter briefly named “the compositions of the invention”.

A macrolide T-cell immunomodulator or immunosuppressant is to be understood herein as being a T-cell immunomodulator or T-cell immunosuppressant which has a macrocyclic compound structure including a lactone or lactam moiety. While it preferably has at least some T-cell immunomodulating or immunosuppressant activity, it may also exhibit concomitantly or predominantly further pharmaceutical properties, such as anti-inflammatory activity.

A retinoid is to be understood herein as being retinoic acid or a compound structurally related to retinoic acid, either natural or synthetic.

The compositions of the invention may be adapted for systemic, e.g. oral or intravenous, or, preferably, for topical use; preferably they are adapted for epicutaneous use. They are useful for the known indications of the particular active agents incorporated therein. They are particularly indicated for use in dermatological diseases, e.g. dermatological diseases which have an inflammatory component or involve inflammatory complications, such as atopic dermatitis, acne, psoriasis, skin aging, sun damage, post-peel erythema and stretch marks and for use in improving the tolerability of retinoid formulations used for the treatment of e.g. skin aging and sun damage, post-peel erythema and stretch marks.

A suitable macrolide T-cell immunomodulator or immunosuppressant is for example an FKBP12-binding calcineurin inhibitor or mitogen-activated kinase modulator or inhibitor, in particular an asco- or rapamycin. It preferably is an ascomycin. While the macrolide preferably has at least some calcineurin- or mitogen-activated kinase modulating or inhibiting activity, it may also exhibit concomitantly or predominantly further pharmaceutical properties, such as antiinflammatory activity. It preferably is a compound, e.g. an ascomycin, having rather long-acting activity relatively to other members of the same structural class, e.g. it is metabolically degraded slowly to inactive products.

An asco- or rapamycin is to be understood as asco- or rapamycin as such, or a derivative thereof. An asco- or rapamycin derivative is to be understood as being an antagonist, agonist or analogue of the parent compound which retains the basic structure and modulates at least one of the biological, for example immunological properties of the parent compound.

An “anti-inflammatory ascomycin derivative” is defined herein as an ascomycin derivative that exhibits pronounced anti-inflammatory activity in e.g. animal models of allergic contact dermatitis but has only low potency in suppressing systemic immune response, namely, which has a minimum effective dose (MED) of up to a concentration of about 0.04% w/v in the murine model of allergic contact dermatitis upon topical administration, while its potency is at least 10 times lower than for tacrolimus (MED 14 mg/kg) in the rat model of allogeneic kidney transplantation upon oral administration (Meingassner, J. G. et al., Br. J. Dermatol. 137 [1997] 568-579; Stuetz, A. Seminars in Cutaneous Medicine and Surgery 20 [2001) 233-241). Such compounds are preferably lipophilic.

Suitable ascomycins are e.g. as described in EP 184162, EP 315978, EP 323.042, EP 423714, EP 427680, EP 465426, EP 474126, WO 91/13889, WO 91/19495, EP 484936, EP 523088, EP 532089, EP 569337, EP 626385, WO 93/5059 and WO 97/8182; in particular:
ascomycin;
tacrolimus (FK506; Prograf®);
imidazolylmethoxyascomycin (WO 97/8182 in Example 1 and as compound of formula I);
32-O-(1-hydroxyethylindol-5-yl)ascomycin (L-732531) (Transplantation 65 [1998] 10-18, 18-26, on page 11, FIG. 1, and
(32-desoxy,32-epi-N1-tetrazolyl)ascomycin (ABT-281) (J. Invest. Dermatol. 12 [1999] 729-738, on page 730, FIG. 1); preferably:
{ 1R,5Z,9S,12S-[1E-(1R,3R,4R)],13R,14S,17R,18E,21S,23S,24R,25S,27R}-17-ethyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0(4,9)]octacos-5,18-diene-2,3,10,16-tetraone (Example 8 in EP 626385), hereinafter referred to as “5,6-dehydroascomycin”;
{1E-(1R,3R,4R)]1R,4S,5R,6S,9R,10E,13S,15S,16R,17S,19S,20S}-9-ethyl-6,16,20-trihydroxy-4-[2-(4-hydroxy-3-methoxycyclohexyl)-1-methylvinyl]-15,17-dimethoxy-5, 11,13,19-tetamethyl-3-oxa-22-azatricyclo[18.6.1.0(1,22)]heptacos-10-ene-2,8,21,27-tetraone (Examples 6d and 71 in EP 569337), hereinafter referred to as “ASD 732”; and especially
pimecrolimus (INN recommended) (ASM981; Elidel™), i.e. {[1E-(1R,3R,4S)]}1R,9S,12S, 13R,14S,17R,18E,21S,23S,24R,25S,27R}-12-[2-(4-chloro-3-methoxycyclohexyl)-1-methylvinyl]-17-ethyl-1,14-dihydroxy-23,25-dimethoxy-13,19,21,27-tetramethyl-11, 28,dioxa-4-azatricyclo [22.3.1.0(4,9)]octacos-18-ene-2,3,10,16-tetraone, of formula I 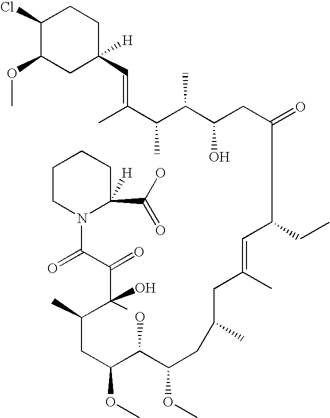
   (Example 66a in EP 427680), hereinafter also referred to as “33-epichloro-33-desoxyascomycin”.

Suitable anti-inflammatory ascomycin derivatives are e.g.: (32-desoxy-32-epi-N1-tetrazolyl)ascomycin (ABT-281); 5,6-dehydroascomycin; ASD 732; and pimecrolimus.

Suitable rapamycins are e.g. as described in U.S. Pat. No. 3,929,992, WO 94/9010 and U.S. Pat. No. 5,258,389, preferably sirolimus (rapamycin; Rapamune®) and everolimus (RAD001; Certican®).

A particularly preferred macrolide T-cell immunomodulator or immunosuppressant is pimecrolimus; it is in free form unless specified otherwise.

A suitable retinold is for example:
acitretin [etretin; (all-E)-9-(4-methoxy-2,3,6-trimethylphenyl)-3,7-dimethyl-2,4,6,8-nona-tetraenoic acid; Soriatane®];
β-carotene (Carotaben®; provitamin A);
etretinate [(all-E)-9-(4-methoxy-2,3,6-trimethylphenyl)-3,7-dimethyl-2,4,6,8-nonatetraenoic acid ethyl ester];
isotretinoin (Accutane®; Roaccutane®);
motretinide [Tasmaderm®; (all-E)-N-ethyl-9-(4-methoxy-2,3,6-trimethylphenyl)-3,7-dimethyl-2,4,6,8-nonatetraenamide];
retinal (retinaldehyde; retinene; vitamin A aldehyde);
retinoic acid (vitamin A acid; tretinoin);
retinol (vitamin A; Retinol®);
retinol palmitate;
tamibaroten;
adapalene (Lorac®; Differin®);
alitretinoin;
bexarotene; or
tazarotene (Zorac®; Tazorac®; synthetic acetylenic retinoid); preferably etretinate, isotretinoin or tazarotene; especially isotretinoin or tazarotene.

Subgroups of compositions of the invention comprise a macrolide T-cell immunomodulator or immunosuppressant, preferably an anti-inflammatory ascomycin derivative as defined above, especially pimecrolimus, in combination or association with a retinoid other than the following retinoids singly or collectively in any number:
etretinate and adapalene; and/or
retinoic acid (vitamin A acid; tretinoin); and/or
acitretin; and/or
tazarotene.

A particularly preferred composition of the invention is pimecrolimus in association or combination with tazarotene.

Preferred for use in the treatment of conditions where inflammation is involved are compositions of the invention wherein one or both components possess some degree of inherent anti-inflammatory activity. The compositions are also particularly beneficial for use where e.g. retinoids can cause some degree of skin inflammation leading to reduced tolerability and local side effects. Particularly preferred are compositions comprising an ascomycin in combination with a retinoid, especially 33-epichloro-33-desoxyascomycin in combination with etretinate, isotretinoin or tazaroten. The inflammatory condition is e.g. eczema, atopic dermatitis, psoriasis, acne, skin aging, sun damage, post-peel erythema or stretch marks.

“Treatment” as used herein includes prevention, namely prophylactic as well as curative treatment.

While retinoids are very effective pharmaceuticals in the treatment of e.g. acne, psoriasis, skin aging, post-peel erythema and stretch marks, their use is often associated with significant side effects such as skin irritation, dry eye, dry skin and keratogenicity. Administering the compositions of the invention allows an improved tolerability profile of retinoid while maintaining efficacy upon e.g. topical administration.

Synergy is e.g. calculated as described in Berenbaum, Clin. Exp. Immunol. 28 (1977) 1, using an interaction term to correct for differences in mechanism between the two drugs, as described in Chou et al., Transpl. Proc. 26 (1994) 3043. The index of synergy is calculated as:

$$\frac{dose of A}{{A}_{E}} + \frac{dose of B}{{B}_{E}} + \frac{( dose of A ) \times ( dose of B )}{{A}_{E} \times {B}_{E}}$$

in which the doses of the compounds A and B represent those used in a particular combination, and A_{E} and B_{E} are the individual doses of A and B respectively giving the same effect. If the result is less than 1, there is synergy; if the result is 1, the effect is additive; if the result is greater than 1, A and B are antagonistic. By plotting an isobologram of dose of A/A_{E} vs. dose of B/B_{E} the combination of maximum synergy can be determined. The synergistic ratio expressed in terms of the ratio by weight of the two compositions at synergistic amounts along the isobologram, especially at or near the point of maximum synergy, can then be used to determine formulations containing an optimally synergistic ratio of the two compounds.

Activity may e.g. be determined in known assay models for testing the pharmacological activity of the individual components of the compositions.

Thus the beneficial effect of the compositions of the invention is apparent in e.g. the acute allergic contact dermatitis (ACD) assay in domestic pigs (Br. J. Dermatol. 137 [1997] 568-576; Br. J. Dermatol. 144 [2001] 788-794) using single drug or combination treatment with pimecrolimus (commercial 1% Elidel® cream) and tazarotene (commercial 0.1% Zorac® gel) administered sequentially:

12 days before elicitation of ACD, 17 male domestic pigs receive 500 μl of 10% 2,4-dinitrofluorobenzene (DNFB) dissolved in dimethylsulfoxide/acetone/olive oil (1:5:3 v/v/v) epicutaneously in divided volumes onto the base of both ears and onto both groins (100 μl/site) for sensitization. The challenge reaction is elicited with 15 μl of DNFB 1.0% on test sites (7 cm² in size) arranged on both sides of the shaved dorsolateral back. Treated and untreated sites are examined 24 hours after the challenge and intensity and extent of erythema and induration are scored on a scale from 0 (absent) to 4 (severw), allowing a combined maximal score of 12 per designated site.

The test sites on the right dorsolateral side are treated with 80 mg formulation twice whereas the left controlateral test sites remain untreated for comparison. The test sites are treated 30 minutes after challenge, followed by the second application at 6 hours of the same drug or, in case of combination, the other drug. Since the responses obtained to combination treatment pimecrolimus/tazarotene, and tazarotene/pimecrolimus are not significantly different, data of both treatment groups are pooled for further evaluation. The results obtained are summarized hereunder, whereby single drug treatment with pimecrolimus, which caused an inhibition of the contact hypersensitivity reaction by 57%, is set to 100% in the Table:

TABLE
ACD in domestic pigs

| Treatment | | | Efficacy¹⁾ | |
|---|---|---|---|---|
| 1st application | 2nd application | n³⁾ | Global mean²⁾ | % |
| pimecrolimus | pimecrolimus | 17 | 3.34 | 100 |
| pimecrolimus | tazarotene | 12 | 3.88 | 116 |
| tazarotene | pimecrolimus | 11 | 3.69 | 111 |
| tazarotene | tazarotene | 12 | 2.96 | 89 |

¹⁾expressed as difference in scores of untreated and treated test sites
²⁾of differences in scores of untreated and treated test sites
³⁾number of animals (2 test sites per animal)

It appears therefrom that single drug treatment with pimecrolimus (100%) proved to be superior by 11% to single-drug treatment with tazarotene (89%). Combination treatments, however, were superior by 11% (tazarotene/pimecrolimus: 111%) and 16% (pimecrolimus/tazarotene: 116%) compared to pimecrolimus/pimecrolimus treatment, or by 22% (tazarotene/pimecrolimus) and 27% (pimecrolimus/tazarotene) in comparison with tazarotene/tazarotene.

The invention also provides products and methods for co-administration of a macrolide T-cell immunomodulator or immunosuppressant, e.g. 33-epichloro-33-desoxy-ascomycin or 5,6-dehydroascomycin, and a retinoid, e.g. etretinate, isotretinoin or tazarotene, at additive/synergistically effective dosages, e.g.:
a method of treatment or prevention of a dermatological disease such as eczema, atopic dermatitis, acne, psoriasis, skin aging, sun damage, post-peel erythema and stretch marks in a subject suffering from or at risk for such condition, comprising co-administering additive/synergistically effective amounts of a composition of the invention;
the use of a macrolide T-cell immunomodulator or immunosuppressant in the manufacture of a medicament for co-administration in additive/synergistically effective amounts with a retinoid;
the use of a retinoid in the manufacture of a medicament for co-administration in additive/synergistically effective amounts with a macrolide T-cell immunomodulator or immunosuppressant;
a kit of parts comprising a macrolide T-cell immunomodulator or immunosuppressant and a retinoid in separate unit dosage forms, preferably wherein the unit dosage forms are suitable for administration of the component compounds in additive/synergistically effective amounts, together with instruction for use, optionally with further means for facilitating compliance with the administration of the component compounds, e.g. a label or drawings;
the use of a macrolide T-cell immunomodulator or immunosuppressant in the manufacture of a pharmaceutical kit which is to be used for facilitating co-administration with a retinoid;
the use of a retinoid in the manufacture of a pharmaceutical kit which is to be used for facilitating co-administration with a macrolide T-cell immunomodulator or immunosuppressant;
a macrolide T-cell immunomodulator or immunosuppressant and a retinoid as a combined pharmaceutical preparation for simultaneous, separate or sequential use, preferably in additive/synergistically effective amounts, e.g. for the treatment or prevention of a dermatological disease such as eczema, atopic dermatitis, acne, psoriasis, skin aging, sun damage, post-peel erythema and stretch marks;
a pharmaceutical composition comprising a macrolide T-cell immunomodulator or immunosuppressant in combination or association with a retinoid, e.g. in additive/synergistically effective amounts, together with at least one pharmaceutically acceptable diluent or carrier, e.g. for use in treatment or prevention of a dermatological disease such as eczema, atopic dermatitis, acne, psoriasis, skin aging, sun damage, post-peel erythema and stretch marks; and
a process for the preparation of a composition of the invention comprising mixing a macrolide T-cell immunomodulator or immunosuppressant and a retinoid, in combination or association with at least one pharmaceutically acceptable diluent or carrier.

By “additive/synergistically effective amounts” is meant an amount of macrolide T-cell immunomodulator or immunosuppressant and an amount of retinoid which are individually below their respective effective dosages for a relevant indication, but which are pharmaceutically active on co-administration, e.g. in an additive/synergistic ratio, for example as calculated above. Furthermore, “synergistically effective amounts” may mean an amount of macrolide T-cell immunomodulator or immunosuppressant and an amount of retinoid which are individually equal to their respective effective dosages for a relevant indication, and which result in a more than additive effect.

The molar amount of macrolide T-cell immunomodulator or immunosuppressant present is from roughly similar to, to significantly less than the amount of retinoid, preferably half as much or less. Additive/synergistic ratios of macrolide T-cell immunomodulator or immunosuppressant to retinoid by weight are thus suitably from about 10:1 to about 1:50, preferably from about 5:1 to about 1:20, most preferably from about 1:1 to about 1:15, e.g. about 1:12.

The compositions of the invention can be administered as a free combination, or the drugs can be formulated into a fixed combination, which greatly enhances the convenience for the patient.

Absolute dosages of the compounds will vary depending on a number of factors, e.g. the individual, the route of administration, the desired duration, the rate of release of the active agent and the nature and severity of the condition to be treated. For example, the amount of active agents required and the release rate thereof may be determined on the basis of known in vitro and in vivo techniques, determining how long a particular active agent concentration in the blood plasma remains at an acceptable level for a therapeutic effect.

For example, in prevention and treatment of a dermatological disease such as eczema, atopic dermatitis, acne, psoriasis, skin aging, sun damage, post-peel erythema and stretch marks, an initial dosage of about 2-3 times the maintenance dosage is suitably administered, followed by a daily dosage of about 2-3 times the maintenance dosage for a period of from one to two weeks, and subsequently the dose is gradually tapered down at a rate of about 5% per week to reach the maintenance dosage. In general, additive/synergistically effective amounts of 33-epichloro-33-desoxyascomycin and retinoid such as tazaroten on oral administration for use in prevention and treatment of eczema, atopic dermatitis, acne, psoriasis, skin aging and sun damage in larger animals, e g. man, are amounts of 33-epichloro-33-desoxyascomycin of up to about 2 mg/kg/day, e.g. from about 0.01 mg/kg/day to about 2 mg/kg/day, preferably about 0.5 mg/kg/day, in combination or co-administration with amounts of retinoid of up to about 50 mg/kg/day, e.g. from about 0.25 mg/kg/day to about 10 mg/kg/day, preferably from about 0.5 mg/kg/day to about 1.0 mg/kg/day, in additive/synergistic ratio, as described. Suitable unit dosage forms for oral co-administration of these compounds thus may contain on the order of from about 0.5 mg to about 100 mg, preferably about 3 mg to about 30 mg of 33-epichloro-33-desoxyascomycin, and from about 10 mg to about 3000 mg, preferably about 50 mg to about 500 mg of retinoid. The daily dosage for oral administration is preferably taken in a single dose, but may be spread out over two, three or four dosages per day. For i.v. administration, the effective dosage is lower than that required for oral administration, e.g. about one fifth the oral dosage.

By “co-administration” is meant administration of the components of the compositions of the invention together or at substantially the same time, e.g. within fifteen minutes or less upon systemic administration, either in the same vehicle or in separate vehicles, so that upon oral administration, for example, both compounds are present simultaneously in the gastrointestinal tract. However, upon topical application, administration of the components may also be separated by an interval of at least several hours, e.g. 6 hours or 12 hours. Preferably, the compounds are administered as a fixed combination.

While the present invention primarily contemplates combination or association of just two pharmaceutically active components, it does not exclude the presence of further active agents, e.g. one further active agent, as far as they do not contradict the purpose of the present invention.

Preferred such further pharmaceutically active components for combination or association are antibacterials.

A suitable antibacterial is for example:
salicylic acid or a salicylic acid derivative, such as: 4-aminosalicylic acid (Apacil®) or 5-aminosalicylic acid (mesalamine; mesalazin; Asacol®) or derivatives thereof, e.g. olsalazin (dimer of mesalamine; 5,5′-azabis[salicylic acid]) or sulfasalazin (5-[p-(2-pyridylsulfamoyl)phenylazo]salicylic acid; Azulfidine®);
a sulfonamide such as sulfacetamide or sulmdiazin;
an antibiotic such as:
a) a penicillin, e.g. penicillin as such or cloxacillin;
b) an amoxicillin; a tetracyclin, e.g. tetracyclin as such, doxycyclin, oxytetracyclin or minocyclin; or a cephalosporin, e.g. ceftazidime or a cephalosporin as described in WO 96/35692, WO 98/43981 and WO 99/48896;
c) a quinolone such as ciprofloxacin, ofloxacin, norfloxacin, levofloxacin or lomefloxacin;
d) a macrolide antibiotic such as erythromycin;
e) clindamycin;
f) chloramphenicol or azidamfenicol (Leukomycin N®); or
g) an aminoglycoside such as gentamycin, kanamycin, neomycin or tobramycin;
h) a polyene such as natamycin;
i) a pseudomonic acid such as mupirocin (pseudomonic acid A);
j) cefiroxim;
k) omiganan (MBI-594; MBI-226) as described in WO 98/07745; or
l) a pleuromutilin;
fusidic acid (ramycin) and derivatives thereof;
metronidazol; or
a polypeptide glycopeptide such as batracin, polymyxin, e.g. polymyxin B, or tyrothricin; preferably a salicylic acid derivative, a penicillin, a quinolone, a macrolide antibiotic or an aminoglycoside; especially sulfasalazin, penicillin, ciprofloxacin, ofloxacin, erythromycin or gentamycin; especially sulfasalazin, ciprofloxacin, ofloxacin, erythromycin or gentamycin; even more preferably ciprofloxacin or erythromycin. It is e.g. active against gram-positive bacteria such as Streptococcus and Staphylococcus or gram-negative bacteria such as Pseudomonas, Escherichia, Enterobacter, Klebsiella, Moraxella and Enterococcus.

The compositions of the invention include compositions suitable for administration by any conventional route, in particular compositions suitable for admninistration either enterally, for example, orally, e.g. in the form of solutions for drinking, tablets or capsules, or parenterally, e.g. in the form of injectable solutions or suspensions; or topically, e.g. for the treatment of inflammatory conditions of the skin or mucosae, e.g. in the form of a dermal cream, ointment, ear drops, mousse, shampoo, solution, lotion, gel, emulgel or like preparation, e.g. in a concentration of from about 0.01% to about 2% by weight of each component, especially in combination or association with penetration enhancing agents, as well as for application to the eye, e.g. in the form of an ocular cream, gel or eye-drop preparation, for treatment of inflammatory conditions of the lungs and airways, e.g. in the form of inhalable compositions, and for mucosal application, e.g. in the form of vaginal tablets.

The compositions of the invention are suitably emulsions, microemulsions, emulsion preconcentrates or microemulsion preconcentrates, or solid dispersions, especially water-in-oil microemulsion preconcentrates or oil-in-water microemulsions, comprising the macrolide T-cell immunomodulator or immunosuppressant and the retinoid in a synergistic ratio.

The compositions of the invention can be prepared in conventional manner, e.g. by mixing a macrolide T-cell immunomodulator or immunosuppressant and a retinoid, in combination or association with at least one pharmaceutically acceptable diluent or carrier.

The active agent components may be in free form or pharmaceutically acceptable salt form as appropriate.

The following Examples illustrate the invention. The compounds are in free, i.e. neutral or base form unless specified otherwise.

### EXAMPLE 1

### Cream

| Component | Amount (g) |
|---|---|
| 33-Epichloro-33-desoxyascomycin | 1.00 |
| tazarotene | 0.10 |
| triglycerides, medium chain | 15.00 |
| oleyl alcohol | 10.00 |
| sodium cetylstearyl sulfate | 1.00 |
| cetyl alcohol | 4.00 |
| stearyl alcohol | 4.00 |
| glyceryl monostearate | 2.00 |
| benzyl alcohol | 1.00 |
| propylene glycol | 5.00 |
| citric acid | 0.05 |
| sodium hydroxide | * |
| water | ad 100.0 |

* amount required to adjust pH to 5.5

The preparation is according to conventional manufacturing procedures for an emulsion. The drug substances are added to the heated homogeneous oily phase which contains triglycerides medium chain, oleyl alcohol, sodium cetylstearyl sulfate, cetyl alcohol, stearyl alcohol and glyceryl monostearate. In parallel, the water phase containing the remaining ingredients is heated at the same temperature as the oily phase. The oily phase is added to the water phase and homogeneisation is performed. The resultant cream is cooled to room temperature.

### EXAMPLE 2

### Cream

As for Example 1, whereby 0.05 g isotretinoin is used in place of 0.10 g tazarotene.

### EXAMPLE 3

### Lotion

| Component | Amount (g) |
|---|---|
| 33-Epichloro-33-desoxyascomycin | 1.00 |
| tazarotene | 0.10 |
| oleyl alcohol | 5.00 |
| propylene glycol | 41.00 |
| isopropanol | 52.90 |
| total | 100.00 |

1. A pharmaceutical composition comprising 33-epichloro-33-desoxyascomycin in combination or association with a retinoid, together with at least one pharmaceutically acceptable diluent or carrier.

2. A composition according to claim 1 wherein the retinoid is etretinate, isotretinoin or tazarotene.

3. A method of treatment of a dermatological disease such as eczema, atopic dermatitis, acne, psoriasis, skin aging, sun damage, post-peel erythema or stretch marks in a subject suffering from or at risk for such condition, comprising co-administering an additive/synergistically effective amount of a composition according to claim 1.

4. A process for the preparation of a composition according to claim 1 comprising mixing 33-epichloro-33-desoxyascomycin and a retinoid, in combination or association with at least one pharmaceutically acceptable diluent or carrier.

5. A kit of parts comprising 33-epichloro-33-desoxyascomycin and a retinoid in separate unit dosage forms, together with instructions for use.

6. A composition according to claim 1 comprising a further pharmaceutically active agent which is an antibacterial.
